(19) United States
(12) Patent Application Publication
(10) Pub. No.: US 2026/0315403 A1
(43) Pub. Date: Oct. 15, 2026

(54) ANTI-INFLAMMATORY FORMULATIONS AND METHODS OF MAKING AND USE THEREOF

(71) Applicant: Global Trademarks, Inc., Nazareth, PA (US)

(72) Inventor: Kaitlyn Schneider, New York, NY (US)

(21) Appl. No.: 19/636,501

(22) Filed: Apr. 1, 2026

Related U.S. Application Data
(60) Provisional application No. 63/781,681, filed on Apr. 1, 2025.

(51) Int. Cl. A61K 9/51; A61K 9/00; A61K 31/505; A61K 31/575; A61K 35/747; A61K 47/24; A61P 29/00

(52) U.S. Cl. CPC A61K 9/5123; A61K 9/0014; A61K 31/505; A61K 31/575; A61K 35/747; A61K 47/24; A61P 29/00

(57) ABSTRACT

An exosome-like nano vesicle formulation comprising: a neutral lipid; and at least three active ingredients selected from the group consisting of an extract, an osmolyte, and an antioxidant, and methods of use thereof, including methods of treating inflammation, increasing skin hydration, and reducing visible signs of aging in the skin.

### CROSS-REFERENCE TO RELATED APPLICATIONS

The present application claims priority to U.S. Provisional Application No. 63/781,681, filed Apr. 1, 2025, the disclosure of which is incorporated herein by reference in its entirety.

### BACKGROUND OF THE INVENTION

The skin serves as the body's first line of defense, constantly exposed to environmental aggressors, including UV radiation, pollution, harsh weather, and pathogens. These external factors, combined with internal triggers such as stress, hormonal fluctuations, and dietary choices, activate the skin's immune response, leading to inflammation. Inflammatory responses are crucial for wound healing and protection against infection; however, when persistent or uncontrolled, they can damage the skin's structural integrity and accelerate signs of aging (Pilkington et al., 2021, International Journal of Molecular Sciences, 22, 6915).

Skin inflammation is typically mediated by pro-inflammatory cytokines—signaling molecules that trigger the immune response—along with other compounds like reactive oxygen species (ROS) and matrix metalloproteinases (MMPs). While acute inflammation helps the skin respond to injury, chronic inflammation, often due to repeated exposure to stressors, disrupts the skin's natural balance and contributes to long-term damage. This “silent inflammation” may not be immediately visible but results in gradual deterioration of the skin's appearance and functionality (Giangreco et al., 2009, Journal of Pathology, 217, 217).

Inflammation in the skin is increasingly recognized as a fundamental factor contributing to a wide range of cosmetic and dermatological concerns, including redness, puffiness, dehydration, sagging, wrinkles, and hyperpigmentation. Often referred to as “inflammaging” or “skinflammation” in scientific literature, chronic, low-grade inflammation accelerates the aging process and exacerbates multiple skin conditions. The term “inflammaging” describes the process by which chronic, low-level inflammation accelerates the aging process at a cellular level. This phenomenon is influenced by both external and internal factors, such as UV exposure, pollution, and psychological stress. Prolonged exposure to these factors initiates a cycle of inflammation and oxidative stress, leading to cumulative cellular damage (Franceschi et al., 2000, Annals of the New York Academy of Sciences, 908, 244).

Oxidative stress from ROS plays a critical role in inflammation-driven skin aging. ROS are reactive molecules generated as a byproduct of cellular metabolism and environmental exposure (e.g., UV rays, pollution). They cause direct damage to cellular components, including DNA, lipids, and proteins. This damage prompts an inflammatory response, which releases more ROS, creating a self-sustaining cycle of oxidative stress and inflammation (Kohen et al., 2001, FASEB Journal, 15, 1456). This vicious cycle ultimately contributes to visible signs of aging, such as wrinkles, sagging, and uneven pigmentation.

Inflammation is a primary driver of redness and sensitivity in the skin. When the skin's immune cells, such as keratinocytes and macrophages, detect potential threats, they release pro-inflammatory cytokines (e.g., interleukin-1, interleukin-6, and tumor necrosis factor-alpha). These cytokines cause blood vessels to dilate, increasing blood flow to the affected area, which manifests as redness. Prolonged exposure to irritants or environmental stressors can lead to chronic redness and conditions like rosacea (Steinhoff et al., 2011, Journal of Clinical Investigation, 121, 3827).

Inflammatory responses also contribute to puffiness, particularly around sensitive areas like the eyes. Inflammation increases vascular permeability, allowing fluid to accumulate in the tissue, resulting in swelling or puffiness. This effect is further exacerbated by lifestyle factors, such as lack of sleep, high salt intake, and allergies, which can promote localized inflammation and fluid retention in the skin (Peck et al., 2016, Journal of Investigative Dermatology, 132, 577).

Inflammation disrupts the skin's barrier function, increasing transepidermal water loss (TEWL) and leading to dehydration. Pro-inflammatory cytokines weaken the lipid matrix that holds skin cells together, making it easier for moisture to escape and irritants to penetrate the skin. This compromised barrier not only results in a lack of hydration but also makes the skin more prone to dryness, irritation, and sensitivity (Goleva et al., 2013, Clinical Reviews in Allergy and Immunology, 44, 143).

Chronic inflammation accelerates the degradation of collagen and elastin fibers, which are essential for maintaining skin firmness and elasticity. Inflammatory mediators like MMPs are activated in response to stress and UV exposure and break down the skin's structural proteins (collagen and elastin). Over time, this process leads to loss of firmness, sagging, and the formation of wrinkles (Quan et al., 2009, Journal of Investigative Dermatology Symposium Proceedings, 14, 20). UV-induced inflammation, in particular, is known to increase MMP expression and collagen degradation, contributing to photoaging.

Wrinkles and fine lines are directly associated with chronic inflammation's impact on collagen and elastin breakdown. Inflammatory cytokines and oxidative stress disrupt the skin's extracellular matrix (ECM), the structural network that supports skin resilience. This leads to a loss of structural integrity, which manifests as fine lines and wrinkles. Additionally, inflammation can impair fibroblast function, reducing collagen synthesis and limiting the skin's ability to repair itself (Fisher et al., 2002, New England Journal of Medicine, 337, 1419).

Inflammatory responses can also trigger melanogenesis, the process by which melanocytes produce melanin. When the skin experiences an inflammatory response—such as acne, injury, or UV exposure—it often results in post-inflammatory hyperpigmentation (PIH). The inflammatory response stimulates melanocytes, causing an overproduction of melanin, which appears as dark spots or uneven pigmentation (Hakozaki et al., 2002, Dermatology Research and Practice). This process is particularly common in individuals with darker skin tones, where hyperpigmentation tends to be more pronounced following inflammation.

The skincare industry has long relied on traditional anti-inflammatory agents to address issues like redness, puffiness, dehydration, and aging. However, these agents often lack the targeted delivery required to effectively treat deeper inflammation at the cellular level. Instead, they work primarily on the skin's surface, resulting in superficial efficacy and often requiring high concentrations to be noticeable. This can lead to unwanted side effects, such as irritation or increased sensitivity, particularly in individuals with reactive or aging skin.

Nanotechnology offers a promising advancement in skincare, specifically through nano-vesicle delivery systems. Nano-vesicles encapsulate active compounds within lipid-based carriers that are structurally similar to cell membranes, allowing for more efficient penetration through the stratum corneum and into deeper layers of the skin. By enhancing bioavailability, stability, and targeted delivery, nano-vesicles overcome several limitations of traditional formulations and offer a more effective approach to treating inflammation-related skin issues (Dreier et al., 2015, Journal of Cosmetic Science, 66, 359). However, existing nano-delivery systems face challenges that limit their effectiveness and usability. These challenges include stability, controlled release, and formulation compatibility.

Thus, there is a need in the art for improved compositions and methods for addressing inflammation of the skin. This invention satisfies this unmet need.

### SUMMARY OF THE INVENTION

The present invention relates to in part an exosome-like nano vesicle formulation comprising: a neutral lipid; and at least three active ingredients selected from the group consisting of an extract, an osmolyte, and an antioxidant.

In some embodiments, the neutral lipid is selected from the group consisting of phosphatidylcholine (PC), phsophatidylserine (PS), phosphatidic acid (PA), phosphatidylethanolamine (PE), phosphatidylglycol (PPG), hydrogenated PC, and combinations thereof.

In some embodiments, the extract is selected from the group consisting of Pseudoalternomonas ferment extract, Lactobacillus kefiranofaciens extract, Aristoloquia clematis extract, Centella asiatica extract, Rosa moschata extract, Echinacea angustifolia extract, Symphytum officinal extract, Equisetum arvense extract, Hypericum perforatum extract, Mimosa tenuiflora extract, Persea gratisima extract, Prunus africanum extract, Tormentilla erecta extract, Aloe vera extract, Calendula Officinalis extract, Hypericum perforatum extract, Chamomilla Recutita extract, Rosmarinus Officinalis extract, and combinations thereof. In some embodiments, the osmolyte is selected from the group consisting of ectoin, sorbitol, glycerol, sarcosine, betaine, taurine, glycine, trimethylamine N-oxide (TMAO), inositol, glycerophosphorylcholine, and combinations thereof. In some embodiments, the antioxidant is Cefiraprotect CLR. In some embodiments, the exosome-like nano vesicle formulation comprises three active ingredients, and the three active ingredients are ectoin, Pseudoalternomonas ferment extract, and Cefiraprotect CLR.

In some embodiments, the concentration of the neutral phospholipid is between 0.01% (w/w) and 10% (w/w). In some embodiments, one of the at least three active ingredients is ectoin. In some embodiments, one of the at least three active ingredients is ectoin, and the concentration of ectoin is between 0.01% (w/w) and 5% (w/w). In some embodiments, one of the at least three active ingredients is Pseudoalternomonas ferment extract. In some embodiments, one of the at least three active ingredients is Pseudoalternomonas ferment extract, and the concentration of Pseudoalternomonas ferment extract is between 0.01% and 5% (w/w). In some embodiments, one of the at least three active ingredients is Cefiraprotect CLR. In some embodiments, one of the at least three active ingredients is Cefiraprotect CLR, and the concentration of Cefiraprotect CLR is between 0.01% (w/w) and 5% (w/w). In some embodiments, the exosome-like nano vesicle formulation further comprises beta-sitosterol. In some embodiments, the exosome-like nano vesicle formulation further comprises a component selected from the group consisting of mannitol, pentylene glycol, glycerin, propanediol, polyglycerol-10 laurate, xanthan gum, citric acid, and combinations thereof.

In some embodiments, the exosome-like nano vesicle formulation is in the form of a liquid, solution, emulsion, suspension, triturate, jelly, foam, paste, ointment, face wash, adhesive, cream, serum, milk, lotion, salve, oil, butter, gel, or balm. In some embodiments, the neutral phospholipid forms exosome-like nano vesicles having an average size between 10 nm and 200 nm.

The present invention further relates to in part a method of treating inflammation of the skin comprising administering to the skin the exosome-like nano vesicle formulation, a method of increasing skin hydration comprising administering to the skin the exosome-like nano vesicle formulation, and a method of reducing visible signs of aging in the skin comprising administering to the skin the exosome-like nano vesicle formulation.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a representative flowchart for the production of the instant formulation.

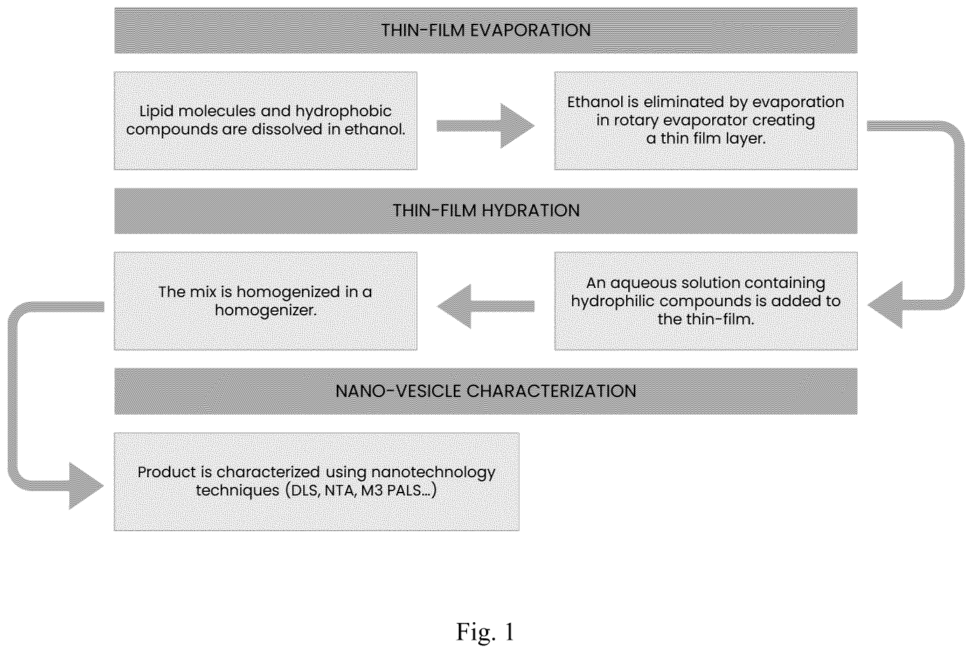

FIG. 2 depicts exemplary images showing reduction of wrinkles in before and after images using Signature Sereme over 6 weeks.

### DETAILED DESCRIPTION

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Exosome-Like Nano Vesicle Formulations

The present invention provides, in part, an exosome-like nano vesicle formulation comprising a neutral lipid and an active ingredient. In some embodiments, the exosome-like nano vesicle formulation comprises: a neutral lipid; and at least three active ingredients selected from the group consisting of an extract, an osmolyte, and an antioxidant. In certain embodiments, the exosome-like nano vesicle formulation is an anti-inflammatory exosome-like nano vesicle formulation. In some embodiments, the exosome-like nano vesicle formulation is uniquely suited to deliver an active ingredient which reduces inflammation in a subject in need thereof. In some embodiments, exosome-like nano vesicle formulation allows the anti-inflammatory active ingredients to be effectively delivered several layers into the skin or dermis.

In certain embodiments, the present formulation provides a delivery vehicle as means of delivering or introducing an active ingredient into a cell of interest. In some embodiments, an exemplary delivery vehicle is an exosome-like nano vesicle or a vesicle.

The delivery vehicles, exosome-like nano vesicles, and vesicles of the present invention are not limited to any particular structure in solution. For example, they may be present in a bilayer structure, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about-20° C. Chloroform is used as the only solvent since it is more readily evaporated than methanol. “Exosome-like nano vesicle” is a generic term encompassing a variety of single and multilamellar lipid vehicles formed by the generation of enclosed lipid bilayers or aggregates. Exosome-like nano vesicles can be characterized as having vesicular structures with a phospholipid bilayer membrane and an inner aqueous medium. Multilamellar exosome-like nano vesicles have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers.

As used herein, the terms “neutral lipid” or “neutral phospholipid” are used interchangeably herein and refer to any one of a number of lipid species known in the art that exist in either an uncharged or neutral zwitterionic form at physiological pH. Exemplary neutral phosphilipids include, but are not limited to, phosphatidylcholines, or lecithin, diacylphosphatidylcholines, diacylphosphatidylethanolamines, ceramides, sphingomyelins, dihydro sphingomyelins, cephalins, and cerebrosides. In certain embodiments, the neutral lipid forms an exosome-like nano vesicle. In some embodiments, the neutral lipid forms a vesicle.

In some embodiments, the neutral lipid is selected from the group consisting of phosphatidylcholine (PC), phsophatidylserine (PS), phosphatidic acid (PA), phosphatidylethanolamine (PE), phosphatidylglycol (PPG), hydrogenated PC, and combinations thereof.

In certain embodiments, the active ingredients used in the instant formulations are anti-inflammatory and serve to reduce inflammation in the skin or dermis. In certain embodiments, the active ingredients used in the instant formulations are anti-aging and serve to reduce the appearance of aging in the skin or dermis such as wrinkling, sagging, and loss of volume. Exemplary active ingredients in the formulation include, but are not limited to, natural and synthetic antioxidants, extracts derived from plants, bacterium, or mixtures thereof, and natural and synthetic osmolytes.

The active ingredients of the present invention may be encapsulated in the aqueous interior of an exosome-like nano vesicle, interspersed within the lipid bilayer of an exosome-like nano vesicle, entrapped in am exosome-like nano vesicle, complexed with am exosome-like nano vesicle, dispersed in a solution containing a lipid, mixed with a lipid, combined with a lipid, contained as a suspension in a lipid, or otherwise associated with a lipid.

In certain embodiments, the exosome-like nano vesicles are spherical. In some embodiments, the average diameter of the exosome-like nano vesicles is between about 10 nm to about 500 nm, about 10 nm to about 450 nm, about 10 nm to about 400 nm, about 10 nm to about 350 nm, about 10 nm to about 300 nm, about 10 nm to about 250 nm, about 10 nm to about 200 nm, about 10 nm to about 150 nm, about 10 nm to about 100 nm, about 10 nm to about 50 nm, about 20 nm to about 500 nm, about 30 nm to about 500 nm, about 40 nm to about 500 nm, about 50 nm to about 500 nm, about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 100 nm to about 450 nm, about 100 nm to about 400 nm, about 100 nm to about 350 nm, about 100 nm to about 300 nm, about 100 nm to about 250 nm, about 100 nm to about 200 nm, about 100 nm to about 150 nm, about 50 nm to about 100 nm, about 100 nm to about 150 nm, about 150 nm to about 200 nm, or at least about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 300 nm, about 400 nm, and about 500 nm.

In some embodiments, the exosome-like nano vesicles have a polydispersity index (PDI) of less than about 0.1, about 0.2, about 0.3, about 0.4, about 0.5, about 0.6, about 0.7, about 0.8, about 0.9, or about 1.0.

Exemplary antioxidants that can be used in the present formulation include vitamin A and the like as well as their derivatives and salts such as retinol, dehydroretinol, retinol acetate, retinol palmitate, retinal, retinoic acid, and vitamin A oil; carotenoid and the like as well as their derivatives such as α-carotene, β-carotene, γ-carotene, cryptoxanthin, astaxanthin, and fucoxanthin; vitamin B and the like as well as their derivatives and salts such as pyridoxine, pyridoxal, pyridoxal-5-phosphate, and pyridoxamine; vitamin D and the like as well as their derivatives and salts such as ergocalciferol, cholecalciferol, and 1,2,5-dihydroxy cholecalciferol; vitamin D and the like as well as their derivatives and salts such as α-tocopherol, β-tocopherol, γ-tocopherol, δ-tocopherol, α-tocotrienol, β-tocotrienol, γ-tocotrienol, δ-tocotrienol, tocopherol acetate, and tocopherol nicotinate; trolox and its derivatives and salts; dihydroxytoluene, butylhydroxytoluene, butylhydroxyanisole, dibutylhydroxytoluene, α-lipoic acid, dehydrolipoic acid, glutathione, and their derivatives and salts; uric acid; erythorbic acid and its derivatives and salts such as erythorbic acid and sodium erythorbate; gallic acid and its derivatives and salts such as gallic acid and propyl gallate; rutin and its derivatives and salts such as rutin and α-glycosyl rutin; triptophan and its derivative and salts; histidine and its derivatives and salts; cysteine and its derivatives and salts such as N-acetylcysteine, N-acetylhomocysteine, N-octanoyl cysteine, and N-acetylcysteine methyl ester; cystine and its derivatives and salts such as N,N′-diacetylcystine dimethyl ester, N,N′-dioctanoylcystine dimethyl ester, and N,N′-dioctanoylhomocystine dimethyl ester; carnosine and its derivatives and salts; homocarnosine and its derivatives and salts; anserine and its derivatives and salts; carcinine and its derivatives and salts; dipeptides or tripeptide derivatives and their salts containing histidine and/or triptophan and/or histamine; flavonoids such as flavanone, flavone, anthocyanin, anthocyanidin, flavonol, quercetin, quercitrin, myricetin, fisetin, hamamelitannin, catechin, epicatechin, gallocatechin, epigallocatechin, epicatechin gallate, and epigallocatechin gallate; tannic acid, caffeic acid, ferulic acid, protocatechuic acid, chalkone, orizanol, carnosol, sesamol, sesamin, sesamolin, zingerone, curcumin, tetrahydrocurcumin, clovamide (caffeoyldopa), deoxyclovamide, shogaol, capsaicin, vanillamide, ellagic acid, bromphenol, flavoglaucin, melanoidin, riboflavin, riboflavin butyrate, riboflavin mononucleotide, flavin adenine dinucleotide, ubiquinone, ubiquinol, mannitol, bilirubin, cholesterol, ebselen, selenomethionine, ceruloplasmin, transferrin, lactoferrin, albumin, superoxide dismutase, catalase, glutathione peroxidase, metallothionein, thioredoxin, thiotaurine, O-phosphono pyridoxyliden rhodamine, and N-(2-hyroxybenzyl) amino acid, and their derivatives and salts; N-(4-pyridoxylmethylene) amino acid, and its derivatives and salts; and others such as lysine, 1-methionine, proline, silymarin, tea extract, grape bark/seed extract, melanin, and rosemary extract. In some embodiments, the antioxidant is Cefiraprotect CLR.

Examples of plant extracts include extracts of plants such as Cynara scolymus, Euphrasia officinalis, Pilea mongolica, Elaeagnus umbellata, Agrimonia eupatoria, Agni fruit, Akebia quinata, fruit of Euterpe oleracea, Withania somnifera, Thujopsis dolabrata, Acacia catechu, Quercus variabilis, Gynostemma pentaphyllum, Pimpinella anisum L., Hamamelis virginiana, Alistin, Epimedium grandiflorum var. thunbergianum, Fallopia japonica (Houtt.) Ronse Decr., Ginkgo biloba, Pyrola japonica, Fragaria, Lilium tenuifolium Fischer, Gloiosiphonia capillaris, Ficus erecta, bark of Tabebuia spp., Acer palmatum Lilium maculatum Thunb., Foeniculum vulgare, Hamamelis virginiana, Gaultheria procumbens, Curcuma longa Linn, Prunus mume, Quercus salicina, Epimedium brevicornum Maxim, Rosa multiflora, Echinacea angustifolia, Eleutherococcus senticosus, Cytisus scoparius, Gentianella alborosea (Gilg) Fabris, Sambucus nigra, Ilex paraguariensis, Borassus flabellifer, Phellodendron amurense, Coptis japonica (Thunb.) Makino, Silybum marianum, Avena sativa, Cardamine scutata, Salsola komarovii, Lilium rubellum, Lapsana apogonoides, Lilium lancifolium, Origanum vulgare, Citrus sinensis, seed of Cucurbita, bark of Erythroxylum catuaba, Lilium speciosum, Paullinia cupana, Hibiscus sabdariffa, Asarum nipponicum, Tussilago farfara, Platycodon grandiflorus, Rumex japonicus, Clerodendron trichotomum, Mallotus philippinensis, Lilium medeoloides A. Gray, Juglans, Vaccinium vitis-idaea L., Monochoria vaginalis var. plantaginea, Eisenia arborea Areschoug, Punica granatum, Ipomoea batatas, Smilax regelii Killip & Morton, Crataegus cuneata, Pellionia minima, Perilla frutescens var. crispa, Psophocarpus tetragonolobus (L.) D.C., Filipendula multijuga, Paeonia lactiflora, Rheum palmatum, Aster scaber, Chenopodium album, Aster ageratoides ssp. leiophyllus, Plantago major, Centella asiatica, Fagopyrum esculentum, Lilium formosanum A. Wallace, Cardamine scutata, Davilla rugosa, Lilium nobilissimum Makino, Cordia salicifolia, Syzygium aromaticum Merr. et Perry, Cassia angustifolia, Centella asiatica (L.) Urb., Bellis perennis, Anethum graveolens, Harpogophytum procumbens, Farfugium japonicum (L.) Kitam, Lilium longiflorum, root of Harpogophytum procumbens, Rubus suavissimus S. Lee, Prunus persica, Houttuynia cordata, Rosa roxburghii, leaves of Eucommia ulmoides, Solanum lycopersicum, Potentilla tormentilla Schrk (Rosaceae), Rumex crispus, Sambucus racemosa subsp. sieboldiana, Trapaeolum majus Linn., Aster microcephalus var. ovatus, Mentha, Lilium longiflorum, Lespedeza sp., Colocasia gigantea, Passiflora incarnata, Passiflora edulis Sims, roots of Pfaffia, Artocarpus heterophyllus, Prunus cerasoides var. campanulata, Helianthus annuus, Elatostema japonicum var. japonicum, Lilium concolor, Lapsana apogonoides, Areca catechu, Petasites japonicus (Siebold et Zucc.), Maxim, Lablab purpureus (L.) Sweet, Cimicifuga racemosa, Actaea racemosa, Crassocephalum crepidioides, Actinidia polygama, Machilus odoratissima, Pinus, Lithocarpus edulis, Llex paraguariensis, Lilium candidum, Silybum marianum, Staphylea bumalda, Stauntonia hexaphylla, Nemacystus decipiens, Salicaceae, Hemerocallis fulva var. kwanso, Annona montana Macfady, Lilium auratum, Ipomoea aquatica, Aster yomena, seeds of Litchi chinensis, Lilium regale, Pleioblastus linearis, Rubus rosaefolius, unripe fruit of Malus pumila, flowers of Tilia cordata, Aspalathus linearis, Lactuca sativa, Citrus limon, Cymbopogon citratus, Thymus×citriodorus, Aloysia citrodora, Melissa officinalis, Rosa canina, flower buds of Rosa damascena, Rosmarinus officinalis L., Rosa×centifolia L., Laurus nobilis, Aerides rosea, Haematoxylum campechianum, Sanguisorba officinalis, Astragalus sinicus, leaves of Diospyros kaki Thunberg, leaves of Glycyrrhiza uralensis, episperms of Glycine max, seeds of Oryza sativa subsp. javanica, leaves of Alpinia zerumebet, Lilium tenuifolium Fischer, Salix sp., leaves of Eucommia ulmoides, and leaves of Angelica keiskei; extracts of sea algae such as Gloiopeltis complanata (Harvey) Yamada, Gloiopeltis furcata (Postels et Ruprecht) J. Agardh, and Gloiopeltis tenax (Turner) J. Agardh; extracts of raw coffee beans, lees of liquor distilled from sweet potatoes, mycelia of Agaricus blazei, etc.; equol; isoflavone; 1,4-anthraquinone; 1-amino-2-hydroymethyl anthraquinone; 4-aminophenol; 1,3,5-trihydroxybenzene, kojic acid; 3,4-dihydroxy-phenylacetate; caffeine acid; ifenprodil; 6-hydroxy-2,5,7,8-tetramethylchroman-2-carboxylate; 6-hydroxyindole; 7-hydroxy-4,6-dimethylphthalide; α-lipo acid, 4-hydroxychalcone; protein hydrolyzates of pearls; aminoguanidine, erythrosine sodium L-ergothioneine, resveratrol; hydroxystilbenes such as 3,3′,5′5-tetrahydroxystilbene; oxindole; carnosine; salicylic acid; salsolinol hydrobromide; sinapinic acid; tocopherylnicotinate; nicotinic-acid amide; nordihydroguaiaretic acid; proanthocyanin; mannitol; hydrolyzed casein; hydrolysable tannin; catechol; chlorogenic acid and derivatives thereof such as chlorogenic acid, isochlorogenic acid, neochlorogenic acid, cryptochlorogenic acid, and feruloyl caffeoylquinic acid; and others such as leucocyanidin, prunin, procyanidol oligomer, and glycosyl rutin. Among the above ingredients, the following can be more suitably used in combination with the L-ascorbic acid, derivatives thereof, and their salts used in the present invention to effectively enhance anti-wrinkle action, anti-small wrinkle action, skin-whitening action, anti-blemish action, and anti-sagging action, exerted by the L-ascorbic acid, derivatives thereof, and their salts: Extracts of Withania somniferous, Thujopsis dolabrata, Acacia catechu, Ginkgo biloba, Gaultheria procumbens, Phellodendron amurense, Citrus sinensis, seed of Cucurbita, bark of Erythroxylum catuaba, Mallotus philippinensis, glucosyl rutin, Vaccinium vitis-idaea L., Perilla frutescens var. crispa, Fagopyrum esculentum, Davilla rugosa, Houttuynia cordata, Elatostema japonicum var. japonicum, Crassocephalum crepidioides, Actinidia deliciosa, Pleioblastus linearis, Rubus croceacanthus, unripe fruit of Malus pumila, leaves of Diospyros kaki Thunberg, leaves of Glycyrrhiza glabra (licorice), leaves of Alpinia zerumbet, extracts of Eucommia ulmoides, equol, isoflavone, ifenprodil, protein hydrolyzates of pearls, catechol, caffeic acid, and prunin. Among the above plant extracts having a glycation-inhibitory action, suitably used are extracts of plants of the family Cupressaceae such as Thujopsis dolabrata, those of the family Ginkgoaceae such as Ginkgo biloba, those of the family Rutaceae such as Phellodendoron amurense Ruprecht, Citrus sinensis, and Citrus limon, those of the family Labiatae such as Perilla frutescens var. crispa and Rosmarinus officinalis L., those of the family Saururaceae such as Houttuynia cordata, and those of the family Eucommiaceae such as Eucommia ulmoides.

In some embodiments, the extract is selected from the group consisting of Pseudoalternomonas ferment extract, Lactobacillus kefiranofaciens extract, Aristoloquia clematis extract, Centella asiatica extract, Rosa moschata extract, Echinacea angustifolia extract, Symphytum officinal extract, Equisetum arvense extract, Hypericum perforatum extract, Mimosa tenuiflora extract, Persea gratisima extract, Prunus africanum extract, Tormentilla erecta extract, Aloe vera extract, Calendula Officinalis extract, Hypericum perforatum extract, Chamomilla Recutita extract, Rosmarinus Officinalis extract, and combinations thereof. In some embodiments, the formulation comprises Pseudoalternomonas ferment extract.

Exemplary osmolytes include, but are not limited to, ectoin, sorbitol, glycerol, sarcosine, betaine, taurine, glycine, trimethylamine N-oxide (TMAO), inositol, glycerophosphorylcholine, proline, sucrose and combinations thereof. In some embodiments, the formulation comprises ectoin.

In certain embodiments, the formulation further comprises radical scavengers, peroxide scavengers, reactive oxygen scavengers, or any combination thereof. Exemplary compounds include, but are not limited to, benzoic acid, methionine, ascorbic acid, pyruvic acid, catalases, peroxidase, vitamin E, vitamin C, vitamin A, derivatives thereof, and any combination thereof. In some embodiments, the formulation further comprises isomalt. In some embodiments, the formulation excludes isomalt.

In certain embodiments, any component (e.g., active ingredient) described of the present invention is present in the instant formulation in a concentration of between about 0.1% (w/w) and about 100% (w/w), 0.1% (w/w) and about 99% (w/w), 0.1% (w/w) and about 90% (w/w), 0.1% (w/w) and about 80% (w/w), 0.1% (w/w) and about 70% (w/w), 0.1% (w/w) and about 60% (w/w), 0.1% (w/w) and about 50% (w/w), 0.1% (w/w) and about 40% (w/w), 0.1% (w/w) and about 30% (w/w), 0.1% (w/w) and about 20% (w/w), 0.1% (w/w) and about 10% (w/w), about 0.1% (w/w) and about 9% (w/w), about 0.1% (w/w) and about 8% (w/w), about 0.1% (w/w) and about 7% (w/w), about 0.1% (w/w) and about 6% (w/w), about 0.1% (w/w) and about 5% (w/w), about 0.1% (w/w) and about 4% (w/w), about 0.1% (w/w) and about 3% (w/w), about 0.1% (w/w) and about 2% (w/w), about 0.1% (w/w) and about 1% (w/w), about 0.5% (w/w) and about 10% (w/w), about 0.5% (w/w) and about 9% (w/w), about 0.5% (w/w) and about 8% (w/w), about 0.5% (w/w) and about 7% (w/w), about 0.5% (w/w) and about 6% (w/w), about 0.5% (w/w) and about 5% (w/w), about 0.5% (w/w) and about 4% (w/w), about 0.5% (w/w) and about 3% (w/w), about 0.5% (w/w) and about 2% (w/w), about 0.5% (w/w) and about 1% (w/w), about 1.0% (w/w) and about 10% (w/w), about 1.0% (w/w) and about 9% (w/w), about 1.0% (w/w) and about 8% (w/w), about 1.0% (w/w) and about 7% (w/w), about 1.0% (w/w) and about 6% (w/w), about 1.0% (w/w) and about 5% (w/w), about 1.0% (w/w) and about 4% (w/w), about 1.0% (w/w) and about 3% (w/w), about 1.0% (w/w) and about 2% (w/w), about 1.0% (w/w) and about 1.5% (w/w), about 1.5% (w/w) and about 2% (w/w), or at least about 0.01% (w/w), about 0.1% (w/w), about 0.2% (w/w), about 0.3% (w/w), about 0.4% (w/w), about 0.5% (w/w), about 0.6% (w/w), about 0.7% (w/w), about 0.8% (w/w), about 0.9% (w/w), about 1% (w/w), about 1.5% (w/w), about 2% (w/w), about 3% (w/w), about 4% (w/w), about 5% (w/w), about 6% (w/w), about 7% (w/w), about 8% (w/w), about 9% (w/w), or about 10% (w/w).

In certain embodiments, the formulation comprises a membrane stabilizer. Examples of membrane stabilizers include, but are not limited to, cholesterol and beta-sitosterol.

In certain embodiments, the formulation comprises a component selected from the group consisting of mannitol, pentylene glycol, glycerin, propanediol, polyglycerol-10 laurate, xanthan gum, citric acid, and combinations thereof.

In some embodiments, the exosome-like nano vesicle formulation is in the form of a liquid, solution, emulsion, suspension, triturate, jelly, foam, paste, ointment, face wash, adhesive, cream, serum, milk, lotion, salve, oil, butter, gel, or balm.

In some embodiments, the pH of the formulation is between about 1 and about 14, about 2 and about 14, about 3 and about 14, about 4 and about 14, about 5 and about 14, about 6 and about 14, about 7 and about 14, about 8 and about 14, about 9 and about 14, about 10 and about 14, about 11 and about 14, about 12 and about 14, about 13 and about 14, about 2 and about 13, about 3 and about 12, about 4 and about 11, about 5 and about 10, about 6 and about 10, about 7 and about 10, about 8 and about 10, about 9 and about 10, or at least about 1, about 2, about 3, about 4, about 5, about 6, about 7, about 8, about 9, about 10, about 11, about 12, about 13, or about 14.

### Methods of Use

In one aspect, the invention provides methods of treating inflammation in a subject in need thereof. In some embodiments, the method comprises administering to a subject a formulation of the present invention comprising a neutral lipid and at least three active ingredients as described herein. In some embodiments, the method comprises administering to a subject a formulation of the present invention comprising at least three anti-inflammatory active ingredients.

In some embodiments, the method is used on the skin or the dermis of the subject in need thereof, or is administered topically. In some embodiments, the method results in a reduction of redness, swelling, puffiness, sensitivity, dehydration, sagging, loss of firmness, wrinkles, fine lines, hyperpigmentation, dark spots, visible signs of aging, or any combination thereof. In some embodiments, the method results in a reduction of inflammation of the skin. In some embodiments, the method results in an increase in skin hydration.

In some embodiments, the method comprises administering the formulation in the form of a liquid, solution, emulsion, suspension, triturate, jelly, foam, paste, ointment, face wash, adhesive, cream, serum, milk, lotion, salve, oil, butter, gel, or balm.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Anti-Inflammatory Nano-Vesicle Delivery System for Skincare Formulations

Given that inflammation is the root cause of many cosmetic concerns, addressing it directly can provide a comprehensive approach to skin health and appearance. By targeting the underlying inflammatory pathways, skincare can potentially improve multiple issues simultaneously, rather than addressing them in isolation. This approach not only offers better cosmetic results but also supports long-term skin health by preserving the skin's structural integrity and resilience.

The use of advanced nano-vesicle technology aims to deliver anti-inflammatory compounds deep into the skin to interrupt the inflammatory cascade at its source. By encapsulating ingredients like Ectoin, Pseudoalteromonas Ferment Extract, and Cefiraprotect CLR within nano-vesicles, our formulations ensure that these actives reach the layers of the skin where inflammation begins. This deep, sustained release approach helps reduce inflammation and its associated effects—addressing multiple cosmetic concerns from redness to wrinkles—by treating the root cause rather than just the symptoms.

Traditional skincare solutions have several limitations. Botanical ingredients, such as green tea extract, chamomile, and aloe vera, are commonly used for their anti-inflammatory and antioxidant properties. They work by neutralizing reactive oxygen species (ROS) and reducing pro-inflammatory cytokine production. While these ingredients can soothe the skin, they are generally limited by natural variability, which affects their potency and consistency. Additionally, many botanical compounds are unstable, degrading quickly when exposed to light and air, thus reducing their effectiveness (Vostálová et al., 2009, Journal of Cosmetic Dermatology, 8, 164).

Retinoids like retinol and tretinoin are widely used in anti-aging formulations due to their ability to stimulate collagen production and accelerate cellular turnover. However, they are known for causing irritation, dryness, and increased sensitivity, especially in individuals with delicate or aging skin. While retinoids can help reduce wrinkles and hyperpigmentation, their lack of targeted delivery means that high concentrations are often needed, leading to side effects that limit their usability for many people (Mukherjee et al., 2006, Clinical Interventions in Aging, 1, 327).

Corticosteroids are powerful anti-inflammatory agents used in dermatology for conditions like eczema and psoriasis. They effectively suppress inflammation by inhibiting the production of pro-inflammatory cytokines, but long-term use can result in skin thinning, increased sensitivity, and dependence. As a result, corticosteroids are not suitable for chronic use in cosmetic formulations aimed at addressing aging or pigmentation (Cao et al., 2009, Pharmacology & Therapeutic, 32, 331).

Moisturizers and emollients, containing ingredients like ceramides and hyaluronic acid, are commonly used to support skin hydration and reinforce the skin barrier. By reducing transepidermal water loss (TEWL), they indirectly help to alleviate symptoms of inflammation. However, they do not actively target inflammatory pathways, limiting their effectiveness in treating inflammation-induced aging or hyperpigmentation (Verdier-Sévrain & Bonté, 2007, Journal of Cosmetic Dermatology, 6, 75).

Niacinamide and certain peptides (e.g., palmitoyl tripeptide-1) are increasingly popular for their anti-inflammatory and skin-repair properties. Niacinamide, for example, can reduce redness, improve elasticity, and inhibit pigmentation transfer. However, the lack of a targeted delivery system limits their penetration and efficacy, requiring consistent and prolonged use for noticeable results (Draelos, 2006, Dermatologic Surgery, 32, 903).

These traditional treatments are valuable for surface-level soothing and hydration but lack the capability to penetrate deeply and target the cellular processes that drive inflammation. The need for higher concentrations often increases the risk of irritation, making these treatments suboptimal for managing inflammation and its downstream effects on aging and skin health.

Nano-vesicle delivery systems represent a significant advancement in skincare by addressing the limitations of traditional treatments. Nano-vesicles are lipid-based carriers that encapsulate active compounds, facilitating deeper penetration into the skin. Because they mimic the structure of cell membranes, nano-vesicles can integrate with skin cells, delivering active ingredients more precisely to where they're needed. This targeted approach not only increases bioavailability but also reduces the likelihood of irritation by minimizing the need for high concentrations.

The present invention, EXO Deep Delivery System (DDS) Ectoin, introduces a customized nano-vesicle system designed to address these challenges. By incorporating active ingredients such as Pseudoalteromonas Ferment Extract, Ectoin, and Cefiraprotect CLR, each selected for its specific anti-inflammatory and protective properties, EXO DDS Ectoin offers a stable, compatible, and effective solution for targeting inflammation-driven skin concerns. Key benefits of this system include enhanced stability, controlled release, and broad formulation compatibility.

The present invention is engineered for monodispersity, maintaining a stable particle size that ensures consistent performance and minimizes aggregation. This stability prolongs shelf life and enhances the delivery of active ingredients deep into the skin layers, maximizing efficacy. Further, the nano-vesicle system is optimized for controlled release, delivering active ingredients gradually to ensure sustained therapeutic levels. This prolonged release mechanism provides long-term effectiveness without the need for high concentrations, reducing the potential for irritation and enhancing the product's suitability for sensitive or aging skin. The system is compatible across a wide range of pH levels and formulation types, allowing it to be incorporated into various skincare products, from serums to creams. This versatility makes it ideal for cosmetic, cosmeceutical, and dermo-pharmaceutical applications, enabling a flexible approach to anti-inflammatory skincare.

By addressing the limitations of both traditional treatments and conventional nano-delivery systems, EXO DDS Ectoin offers a targeted, deep-penetrating solution for managing inflammation and its effects on the skin. This system represents a significant step forward in skincare technology, providing a sophisticated approach to tackling the root cause of cosmetic skin concerns rather than merely treating surface symptoms.

The present example provides a cutting-edge nano-vesicle delivery system specifically designed to address chronic skin inflammation, a root cause of aging and various cosmetic concerns. This DDS utilizes exosome-like, lipid-based nano-vesicles to encapsulate three key active ingredients: Ectoin, Pseudoalteromonas Ferment Extract (Luceane™), and Cefiraprotect. Each of these ingredients was selected for its unique anti-inflammatory and protective properties, targeting inflammation at the cellular level.

The nano-vesicle structure mimics cell membranes, allowing for deep and targeted penetration into the skin layers, where chronic inflammation typically originates. The DDS technology enables a controlled, sustained release of active compounds, maintaining a consistent therapeutic level and providing extended anti-inflammatory effects without the need for high concentrations. This prolonged release minimizes irritation, making the system suitable for sensitive skin and enhancing the compatibility across various skincare formulations.

Key benefits of the invention include, but are not limited to, controlled sustained release, comprehensive multi-action solutions, and targeted cellular delivery. The DDS technology delivers active compounds gradually, ensuring prolonged therapeutic action and minimizing the risk of irritation. This controlled release enhances efficacy while supporting long-term skin health. Each active ingredient offers synergistic anti-inflammatory, antioxidant, and reparative properties, addressing multiple concerns such as redness, elasticity loss, and dehydration simultaneously. The nano-vesicles penetrate deeply into the skin, delivering active ingredients to the precise areas where inflammation and damage occur, amplifying their impact at a cellular level.

This invention addresses the limitations of traditional skincare solutions by combining enhanced stability, targeted delivery, and controlled release. This innovative system effectively reduces pro-inflammatory cytokines and oxidative stress, supporting long-term skin health and resilience against environmental aggressors. By focusing on the root cause of inflammation, Miracle Complex offers a comprehensive solution for managing inflammaging and other inflammation-driven skin concerns, representing a significant advancement in the field of cosmeceuticals and anti-aging skincare.

### Product Identification

### Tradename

EXO DDS Ectoin

### Description

PSEUDOALTEROMONAS FERMENT EXTRACT (1.5%), ECTOIN (1%) and CEFIRAPROTECT CLR* (1%) encapsulated in vegan deep release nano-vesicles (DDS-Deep Delivery System) exosome-like to add in cosmetic, cosmeceutical or dermo pharmaceutical formulations.

### INCI

AQUA, MANNITOL, PENTYLENE GLYCOL, PHOSPHATIDYLCHOLINE, GLYCERIN, PSEUDOALTEROMONAS FERMENT EXTRACT, ECTOIN, CEFIRAPROTECT CLR*, POLYGLYCERYL-10 LAURATE, BETA-SITOSTEROL, PROPANEDIOL, XANTHAN GUM, CITRIC ACID

COMPOSITION

| Ingredient | % (w/w) |
|---|---|
| AQUA | Q.S. 100 |
| MANNITOL | 4.30-6.30 |
| PENTYLENE GLYCOL | 4.10-6.10 |
| PHOSPHATIDYLCHOLINE | 4.00-6.00 |
| GLYCERIN | 2.50-3.50 |
| PSEUDOALTEROMONAS FERMENT EXTRACT | 1.00-2.00 |
| ECTOIN | 0.50-1.50 |
| CEFIRAPROTECT CLR | 0.50-1.50 |
| POLYGLYCERYL-10 LAURATE | 0.70-0.90 |
| BETA-SITOSTEROL | 0.20-0.40 |
| PROPANEDIOL | 0.10-0.15 |
| XANTHAN GUM | 0.013-0.017 |
| CITRIC ACID | 0.010-0.014 |

### SPECIFICATIONS

Appearance: Liquid
Color: White
Odor: Characteristic
Size (nm): 50-200
Polydispersity Index: <0.5
pH: 9.0-10.0

### Characterization

Product characterization is based on the particle analysis using nanotechnology characterization techniques such as: Dynamic Light Scattering, Nanoparticle Track Analysis, or Phase Analysis Light Scattering. The primary properties analyzed are average size and size distribution.

### Average Size

Average size of nano-vesicles can be determined using Dynamic Light Scattering (DLS) and Nanoparticle Track Analysis (NTA) techniques. Both are based on the Brownian motion and the light scattering from the particle. They apply the Stokes-Einstein equation to relate diffusion to size (hydrodynamic diameter). Additionally, DLS offers an ensemble measurement, whereas NTA delivers a particle-by-particle measurement.

### Size Distribution

Size distribution was determined using the DLS technique. The distribution is represented by the polydispersity index (PDI), whose values fall in a range between 0 and 1. Values near) represent a monodispersed sample and values near 1 represent a polydispersed sample. It can be considered that values below 0.5 have a good distribution.

### Dispersibility

Product dispersible in aqueous media.

### Incompatibilities

Ethanol concentrations higher than 15%
High concentration of hard detergents
In emulsions prior to product dispersion in aqueous media

### Recommended Dose

1%-10% according to the frequency of application of the final product and the intensity of the effect that you want to achieve.

Exemplary dosages are as follows:
Day Cream: 3%-5%
Serum: 4%-8%
Mask: 3%-7%
Ampoules: 5%-10%
Professional Use: 7%-10%

### Use Instructions

Shake before using. If the product is stored under 12° C., let the product reach room temperature before shaking. At low temperatures reversible changes in viscosity can occur.

Add to bulk during final phase of the production process, ensuring that the temperature does not exceed 40° C. to avoid degradation of the encapsulated molecules.

Maximum Homogenization: 20.000 rpm

Formulation pH: 3-11

### Expiration+Storage

Store in a clean, dark, and refrigerated place (2-8° C.). Do not Freeze

Store for 12 months if product is kept in original sealed container. Once opened, it is recommended to use full package contents at once or repackage excess using nitrogen. Shake before using.

The present example is a cutting-edge nano-vesicle technology designed to address skin inflammation at its root. By utilizing a specialized Deep Delivery System (DDS), this technology enables precise, sustained release of powerful anti-inflammatory and protective agents deep within the skin layers. This targeted approach not only enhances the efficacy of each active ingredient but also minimizes the irritation often associated with surface-level anti-inflammatory treatments, making it ideal for treating both visible inflammation and underlying chronic inflammatory processes that contribute to skin aging and various cosmetic concerns.

The Miracle Complex™ technology encapsulates three key active ingredients, each selected for its unique ability to counteract inflammation and protect the skin from environmental aggressors.

Known for its cell-protective and anti-inflammatory properties, Ectoin is a natural extremolyte that forms a stabilizing “hydro complex” around skin cells. This complex reduces cellular inflammation by preventing environmental stressors, such as

UV radiation and pollution, from disrupting cell membranes. Ectoin has been shown to decrease pro-inflammatory cytokine activity, which helps reduce redness, irritation, and chronic inflammation that can lead to premature aging. By stabilizing cells against oxidative stress, Ectoin effectively interrupts the inflammation cascade at a cellular level, providing both immediate and long-term anti-inflammatory benefits.

This biofermented oligosaccharide extract strengthens mitochondrial function and enhances cell respiration, protecting the skin against oxidative damage. Pseudoalteromonas Ferment Extract counteracts what is termed “hypoxiageing,” which occurs when cells are deprived of adequate oxygen due to chronic inflammation. By enhancing cellular energy (ATP) production and maintaining mitochondrial homeostasis, Pseudoalteromonas Ferment Extract helps regenerate the skin's barrier function, making it more resilient against environmental triggers that provoke inflammation. Additionally, it supports ceramide production, which reinforces the skin barrier and further reduces susceptibility to irritants and allergens, thus lowering inflammatory responses.

Derived from kefir polysaccharides, Cefiraprotect CLR is a powerful antioxidant and anti-inflammatory agent that targets oxidative stress and reactive oxygen species (ROS), key contributors to inflammation. By neutralizing ROS, Cefiraprotect CLR protects skin proteins like collagen and elastin from oxidative degradation, which is commonly exacerbated by inflammatory responses. This ingredient also stabilizes the skin's microbiome, which plays a critical role in maintaining immune balance and reducing chronic inflammation. By mitigating oxidative stress and supporting the skin's natural defenses, Cefiraprotect CLR prevents inflammatory markers from accumulating, thus slowing down skin aging and reducing visible redness and puffiness.

The Deep Delivery System (DDS) used in Miracle Complex™ is specifically designed to enhance the penetration, bioavailability, and stability of these anti-inflammatory agents. The nano-vesicles are lipid-based and structurally similar to exosomes, allowing them to integrate seamlessly with skin cell membranes. This structure is crucial for delivering anti-inflammatory ingredients deep into the skin, where chronic inflammation often originates.

Unlike traditional topical treatments that remain on the skin's surface, the nano-vesicle system ensures that the encapsulated ingredients penetrate the stratum corneum and reach the deeper epidermal and dermal layers. By delivering actives directly to the cells responsible for inflammatory responses, EXO DDS ECTOIN reduces the production of pro-inflammatory cytokines and ROS at the source. This deep penetration minimizes superficial irritation, providing anti-inflammatory benefits precisely where they are needed most.

The DDS nano-vesicles are engineered for controlled, gradual release of active ingredients. This sustained delivery ensures a steady therapeutic level of anti-inflammatory agents over time, allowing the skin to maintain anti-inflammatory activity without the need for high concentrations. By releasing actives slowly, the system minimizes the risk of irritation, even for sensitive or reactive skin types, and provides a prolonged anti-inflammatory effect that supports long-term skin health.

Encapsulating active ingredients within nano-vesicles protects them from premature degradation due to environmental exposure (e.g., light, air). This stability is particularly beneficial for anti-inflammatory agents like Ectoin and Cefiraprotect CLR, which are sensitive to environmental factors. The stable, encapsulated form reduces the likelihood of active ingredient breakdown on the skin's surface, thereby decreasing the potential for irritation. As a result, the skin benefits from potent anti-inflammatory effects without the drawbacks of traditional high-concentration applications.

The Miracle Complex™ technology represents a comprehensive approach to combating inflammation, leveraging the unique anti-inflammatory properties of Ectoin, Pseudoalteromonas Ferment Extract, and Cefiraprotect alongside an advanced nano-vesicle delivery system. By delivering active ingredients directly to the deeper layers of the skin and releasing them gradually, Miracle Complex™ targets inflammation at the cellular level, reducing both visible and underlying inflammatory markers that contribute to aging and skin damage. This system provides a powerful, sustained anti-inflammatory effect that addresses the root cause of many skin concerns, helping to restore skin health, radiance, and resilience.

This innovative technology marks a significant advancement in anti-inflammatory skincare, offering a sophisticated solution for managing inflammaging and inflammation-driven skin concerns.

### Example 2: Clinical Study Report

Objective results Aeston West Proprietary Active were obtained through gene expression analysis of 1% Cellevitae on primary dermal fibroblasts and are as follows:
+1630% increase in growth factor VEGFA gene expression (Fold change=17.3×)
   Impact (What does VEFGA do?)
      Wound healing: stimulates angiogenesis (new blood vessel formation), necessary for tissue regeneration
      Epithelial repair and cell migration: key processes in skin renewal
      Enhancing microcirculation: improves delivery of nutrients and oxygen to the skin
+96% increase in SIRT1 (Sirtuin 1) gene expression (Fold change=1.96×)
   Impact (What does SIRT1 do?)
      Protection against oxidative stress and UV-induced damage
      Collagen preservation by inhibiting MMPs
      Improved mitochondrial function and cell survival
      Promotion of keratinocyte differentiation (skin renewal) over unnecessary proliferation

Immediate results from the Magnolia Cleansing Nectar were observed after 1 use in a study of 43 women age 35-65. Subjective results were as follows:
100% agreed the cleanser glides smoothly on the skin without pulling or tugging
91% agreed that the skin felt nourished and cared for
90% agreed that no irritation was felt around the eyes
88% agreed that skin felt calm and soothed after one use
88% agreed that skin felt clean without tightness or dryness
87% agreed that the skin felt smoother and softer to the touch
87% agreed that the cleanser fully removed daily buildup (SPF, makeup, grime)
87% agreed that the cleanser effective removed waterproof makeup
87% agreed that skin felt replenished and comforted after cleansing
87% agreed that they would recommend the cleanser to someone with sensitive skin
85% agreed that skin felt cleansed without feeling stripped
84% agreed that skin felt deeply hydrated after cleansing
84% agreed that they enjoyed cleansing with the product
83% agreed that they would feel confident using it before a major event
81% agreed that their skin felt prepped for makeup

Immediate results from the Marine Treatment Mist were observed after 1 use in a study of 43 women age 35-65. Subjective results were as follows:
98% agreed that they experienced no irritation or stinging
97% agreed that the mist felt cooling and refreshing on contact
95% agreed that the mist felt soothing on application
94% agreed that the product worked well with their makeup routine
90% agreed that the essence felt lightweight on their skin
89% agreed that the product layered well under other skincare
89% agreed that they would recommend the essence for sensitive skin
87% agreed that the mist replenished moisture and prevented dryness
83% agreed that they would feel confident using it before a major event
82% agreed that skin felt more moisturized after the first spray
82% agreed that skin felt instantly hydrated after use

Objective results were obtained for the Signature Sereme through instrumentation analysis of global facial lines in a study of 33 women age 35-65 over 6 weeks.
100% of subjects showed visible wrinkle reduction in 6 weeks
24% average wrinkle reduction in six weeks

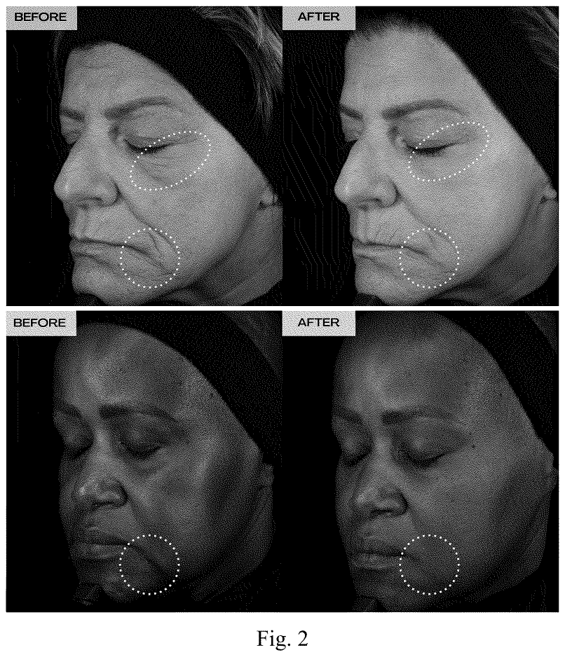

Before and after images are shown in FIG. 2.

Immediate results were observed with Signature Sereme after 1 use in a study of 43 women age 35-65. Subjective results were as follows:
96% agreed the serum delivered both comfort and performance
94% agreed that the product layered well with the rest of their skincare
93% agreed the serum created a smooth base for makeup
92% agreed their skin stayed moisturized throughout the day
89% agreed the hybrid texture felt both rich and breathable
86% agreed skin looked well rested after use
84% agreed skin felt deeply hydrated
83% agreed the serum made skin feel calmer and more stable
82% agreed skin's texture looked and felt smoother

Additional subjective results of Signature Sereme after 1 week with 2× daily use in a study of 43 women age 35-65 were also collected and as follows:
93% agreed skin felt smoother and more even to the touch after one week of use
93% agreed they would trust the serum to deliver lasting results
92% agreed the serum helped reinforce the skin barrier
92% agreed skin felt deeply hydrated
89% agreed the product felt calming and gentle on the skin
86% agreed skin looked brighter and more luminous
84% agreed skin's texture improved after one week of use
83% agreed skin looked healthier and more youthful than it did a week ago
82% agreed skin looked and felt renewed
81% agreed wrinkles looked less deep and defined after one week of use
81% agreed their complexion looked clearer and more refined
80% agreed the serum delivered a noticeable firming effect
80% agreed skin looked and felt plumper

Additional subjective results of Signature Sereme after 1 month with 2× daily use in a study of 33 women age 35-65 were also collected and as follows:
93% agreed the product felt calming and gentle on skin
90% agreed skin felt more lifted and supported
90% agreed skin looked brighter and more luminous
90% agreed skin looked and felt refreshed
90% agreed skin felt smoother and more even to the touch after one month of use
90% agreed skin felt deeply hydrated
87% agreed skin looked and felt plumper
87% agreed skin felt stronger and more resilient
87% agreed serum helped reinforce their skin barrier
87% agreed skin's texture improved after one month of use
84% agreed serum delivered a noticeable firming effect
84% agreed they felt more confident in bare skin after using the serum
84% agreed skin looked healthier and more youthful than it did a month ago
84% agreed skin tone appeared more even after a month of use
84% agreed serum replaced their moisturizer in their routine
84% agreed complexion looked clearer and more refined
84% agreed they would trust the serum to deliver lasting results
84% agreed the appearance of pores looked minimized after a month of use
81% agreed skin felt less reactive or sensitive
81% agreed the product made them feel more youthful and refreshed

Additional claims include that the products are dermatologist tested, safe for sensitive skin, non-irritating, made with clean formulas, cruelty free, clinically tested, vegan, silicone free, and gluten free.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An exosome-like nano vesicle formulation comprising:
a neutral lipid; and
at least three active ingredients selected from the group consisting of an extract, an osmolyte, and an antioxidant.

2. The exosome-like nano vesicle formulation of claim 1, wherein the neutral lipid is selected from the group consisting of phosphatidylcholine (PC), phsophatidylserine (PS), phosphatidic acid (PA), phosphatidylethanolamine (PE), phosphatidylglycol (PPG), hydrogenated PC, and combinations thereof.

3. The exosome-like nano vesicle formulation of claim 1, wherein the extract is selected from the group consisting of Pseudoalternomonas ferment extract, Lactobacillus kefiranofaciens extract, Aristoloquia clematis extract, Centella asiatica extract, Rosa moschata extract, Echinacea angustifolia extract, Symphytum officinal extract, Equisetum arvense extract, Hypericum perforatum extract, Mimosa tenuiflora extract, Persea gratisima extract, Prunus africanum extract, Tormentilla erecta extract, Aloe vera extract, Calendula Officinalis extract, Hypericum perforatum extract, Chamomilla Recutita extract, Rosmarinus Officinalis extract, and combinations thereof.

4. The exosome-like nano vesicle formulation of claim 1, wherein the osmolyte is selected from the group consisting of ectoin, sorbitol, glycerol, sarcosine, betaine, taurine, glycine, trimethylamine N-oxide (TMAO), inositol, glycerophosphorylcholine, and combinations thereof.

5. The exosome-like nano vesicle formulation of claim 1, wherein the antioxidant is Cefiraprotect CLR.

6. The exosome-like nano vesicle formulation of claim 1, wherein the exosome-like nano vesicle formulation comprises three active ingredients, and the three active ingredients are ectoin, Pseudoalternomonas ferment extract, and Cefiraprotect CLR.

7. The exosome-like nano vesicle formulation of claim 1, wherein the concentration of the neutral phospholipid is between 0.01% (w/w) and 10% (w/w).

8. The exosome-like nano vesicle formulation of claim 1, wherein one of the at least three active ingredients is ectoin.

9. The exosome-like nano vesicle formulation of claim 1, wherein one of the at least three active ingredients is ectoin, and the concentration of ectoin is between 0.01% (w/w) and 5% (w/w).

10. The exosome-like nano vesicle formulation of claim 1, wherein one of the at least three active ingredients is Pseudoalternomonas ferment extract.

11. The exosome-like nano vesicle formulation of claim 1, wherein one of the at least three active ingredients is Pseudoalternomonas ferment extract, and the concentration of Pseudoalternomonas ferment extract is between 0.01% and 5% (w/w).

12. The exosome-like nano vesicle formulation of claim 1, wherein one of the at least three active ingredients is Cefiraprotect CLR.

13. The exosome-like nano vesicle formulation of claim 1, wherein one of the at least three active ingredients is Cefiraprotect CLR, and the concentration of Cefiraprotect CLR is between 0.01% (w/w) and 5% (w/w).

14. The exosome-like nano vesicle formulation of claim 1, further comprising beta-sitosterol.

15. The exosome-like nano vesicle formulation of claim 1, further comprising a component selected from the group consisting of mannitol, pentylene glycol, glycerin, propanediol, polyglycerol-10 laurate, xanthan gum, citric acid, and combinations thereof.

16. The exosome-like nano vesicle formulation of claim 1, wherein the exosome-like nano vesicle formulation is in the form of a liquid, solution, emulsion, suspension, triturate, jelly, foam, paste, ointment, face wash, adhesive, cream, serum, milk, lotion, salve, oil, butter, gel, or balm.

17. The exosome-like nano vesicle formulation of claim 1, wherein the neutral phospholipid forms exosome-like nano vesicles having an average size between 10 nm and 200 nm.

18. A method of treating inflammation of the skin comprising administering to the skin the exosome-like nano vesicle formulation of claim 1.

19. A method of increasing skin hydration comprising administering to the skin the exosome-like nano vesicle formulation of claim 1.

20. A method of reducing visible signs of aging in the skin comprising administering to the skin the exosome-like nano vesicle formulation of claim 1.
